(19) United States
(12) Patent Application Publication
(10) Pub. No.: US 2009/0221974 A1
(43) Pub. Date: Sep. 3, 2009

(54) IMPLANTABLE ATRAUMATIC MEDICAL SITE HAVING A SIMPLIFIED DESIGN

(76) Inventor: Pascal Paganon, Serezin du Rhone (FR)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(21) Appl. No.: 12/093,756

(22) PCT Filed: Nov. 16, 2006
(86) PCT No.: PCT/FR2006/002530

§ 371 Date: Jul. 31, 2008

(30) Foreign Application Priority Data
Nov. 16, 2005 (FR) 0511637

(51) Int. Cl. A61M 5/14; B23P 11/00; B32B 7/12

(52) U.S. Cl. 604/244; 29/428; 156/91

(57) ABSTRACT

An implantable device (1) for injecting and/or drawing fluid or an inflatable and/or deflatable compartment of a surgical implant, the device (1) comprising a hollow body (2) and a puncture wall (3) added to the hollow body (2) so that the hollow body (2) and puncture wall (3) delimits a chamber (4). The puncture wall (3) is transfixed by a hollow needle for injection and/or drawing the fluid into the chamber (4), the device (1) comprises a means for fixing (8) the puncture wall to the hollow body (2) so that the hollow body (2), puncture wall (3) and fixing means (8) form a single subassembly (9) and an enclosure (10) essentially covering the puncture wall (3).

### PRIORITY CLAIM

This patent application is a U.S. National Phase of International Patent Application No. PCT/FR2006/002530, filed Nov. 16, 2006, which claims priority to French Patent Application No. 0511637, filed Nov. 16, 2005, the disclosures of which are incorporated herein by reference in their entirety.

### FIELD

The present disclosure relates to devices designed to be inserted under the skin of a human or animal patient for the purpose of being subsequently pierced with a hollow needle through the skin of the patient with a view to injecting substances into the body of the patient and/or to tapping substances from the body of the patient, while limiting reiterated injury to the skin at the same place. Such devices are generally referred to as “implantable sites,” or as “access ports” or as “infusion ports”.

The present disclosure relates more particularly to an implantable device for injecting fluid into and/or for tapping fluid from either an organ or vessel of the body of a human or animal patient, or else an inflatable and/or deflatable compartment of a surgical implant, the device comprising a hollow body and a puncture wall mounted on the hollow body in a manner such that the hollow body and the puncture wall contribute to defining a chamber designed to be put into communication with the organ, vessel or compartment, the puncture wall is designed to be transpierced by a hollow needle with a view to injecting fluid into and/or to tapping fluid from the chamber.

The present disclosure also relates to a method of manufacturing an implantable device for injecting fluid into and/or for tapping fluid from either an organ or vessel of the body of a human or animal patient, or else an inflatable and/or deflatable compartment of a surgical implant, which method comprises mounting a puncture wall on a hollow body, in a manner such that the hollow body and the puncture wall contribute to defining a chamber designed to be put into communication with the organ, vessel or compartment, the puncture wall being designed to be transpierced by a hollow needle with a view to injecting fluid into and/or to tapping fluid from the chamber.

### BACKGROUND

Known implantable sites comprise a housing having a disk-shaped bottom from which there extends a cylindrical side wall whose free ends define a proximal opening. The bottom and the side wall are made of an uninterrupted and rigid material, such as titanium, for example, in order to prevent the bottom and side wall from being transpierced by the puncturing hollow needle. The proximal opening is closed off by a thick self-sealing membrane made of silicone, thereby forming a “septum”.

The rigidness and hardness of the housing, and the non-smooth surface where the housing meets the membrane, make such sites that are known in the prior art traumatic, i.e., when such known sites are inserted in the body of the patient, the known sites can injure the surrounding biological tissue thereby giving rise to a feeling of discomfort or even pain in the patient.

In order to remedy that problem, the present disclosure provides an implantable site in which the septum membrane is merely placed in the proximal opening without any real fastening between the membrane and the housing, a cladding of silicone being molded directly over the housing and fusing with the membrane at the periphery thereof. That one-piece overmolded cladding of silicone in which the housing is embedded makes it possible to give the site the characteristic of being atraumatic. The cladding overlies the side wall of the housing thereby avoiding any direct contact between the biological tissue and the titanium, and the cladding is intimately bonded to the periphery of the septum membrane thereby masking any surface discontinuity where the membrane and the housing meet.

The cladding also performs the function of fastening the membrane to the housing.

Such a site is generally satisfactory because it makes it possible to reduce significantly the risk of tissue injury and also the risk of leakage due to any disengagement of the membrane from the housing.

Unfortunately, those prior art devices are relatively complex to manufacture because, in practice, the operation of overmolding the cladding is particularly difficult to control, in particular, as regards controlling the positioning, the cohesion and the thickness of material at the interface between the septum and the cladding. That difficulty concerning industrial reproducibility tends to slow down the manufacturing rate and requires a qualified and trained workforce.

### SUMMARY

The present disclosure describes several exemplary embodiments of the present invention.

One aspect of the present disclosure provides an implantable device for injecting fluid into or for tapping fluid from either an organ or vessel of the body of a human or animal patient, or an inflatable or deflatable compartment of a surgical implant, the device comprising a) a hollow body; and b) a puncture wall mounted on the hollow body in a manner such that the hollow body and the puncture wall contribute to defining a chamber designed to be put into communication with the organ, vessel, or compartment, the puncture wall is designed to be transpierced by a hollow needle with a view to injecting fluid into or to tapping fluid from the chamber, the device further comprises firstly fastener means for fastening the puncture wall to the hollow body in a manner such that the hollow body, the puncture wall, and the fastener means form a unitary subassembly, and secondly a casing that is distinct from the subassembly and that covers the subassembly so as substantially to overlie the puncture wall, the subassembly and the casing being assembled together to form the device.

Another aspect of the present disclosure provides a method of manufacturing an implantable device for injecting fluid into or for tapping fluid from either an organ or vessel of the body of a human or animal patient, or an inflatable or deflatable compartment of a surgical implant, the method comprising i) mounting a puncture wall on a hollow body in a manner such that the hollow body and the puncture wall contribute to defining a chamber designed to be put into communication with the organ, vessel, or compartment, the puncture wall is designed to be transpierced by a hollow needle with a view to injecting fluid into or to tapping fluid from the chamber, the method further comprising a) fastening the puncture wall to the hollow body by means of fastener means, in a manner such that the puncture wall, the fastener means, and the hollow body form a unitary subassembly; b) fabricating or supplying a casing that is distinct from the subassembly; and c) covering the unitary subassembly in the casing, by assembling together the unitary subassembly and the casing, in a manner such that the casing substantially overlies the puncture wall.

The present disclosure provides an implantable device for injecting and/or tapping fluid that makes it possible to remedy the various drawbacks enumerated hereinabove, and that presents an atraumatic external appearance while also being particularly easy and inexpensive to construct.

One feature of the present disclosure provides an implantable device for injecting and/or tapping fluid that is of particularly simple design, implementing as few parts as possible.

Another feature of the present disclosure provides implantable device for injecting and/or tapping fluid that is particularly well tolerated by the patient.

Still another feature of the present disclosure provides an implantable device for injecting and/or tapping fluid that is particularly robust.

A further feature of the present disclosure provides an implantable device for injecting and/or tapping fluid that minimizes the risk of leakage from the device.

Yet another feature of the present disclosure provides an implantable device for injecting and/or tapping fluid that is particularly strong and reliable.

Another aspect of the present disclosure provides a method of manufacturing an implantable device for injecting and/or tapping fluid that makes it simple, quick, and inexpensive to obtain a reliable implantable device that is atraumatic.

Another feature of the present disclosure provides a method of manufacturing an implantable device for injecting and/or tapping fluid that makes it possible not to use the technique of overmolding.

Features of the present disclosure are thus achieved by means of an implantable device for injecting fluid into and/or for tapping fluid from either an organ or vessel of the body of a human or animal patient, or else an inflatable and/or deflatable compartment of a surgical implant, the device comprising a hollow body and a puncture wall mounted on the hollow body in a manner such that the hollow body and the puncture wall contribute to defining a chamber designed to be put into communication with the organ, vessel or compartment, the puncture wall is designed to be transpierced by a hollow needle to inject fluid into and/or to tap fluid from the chamber, the device further comprising firstly fastener means for fastening the puncture wall to the hollow body in a manner such that the hollow body, the puncture wall and the fastener means form a unitary subassembly, and secondly a casing that is distinct from the subassembly and that covers the subassembly so as substantially to overlie the puncture wall, the subassembly and the casing being assembled together to form the device.

Features of the present disclosure are also achieved by means of a method of manufacturing an implantable device for injecting fluid into and/or for tapping fluid from either an organ or vessel of the body of a human or animal patient, or else an inflatable and/or deflatable compartment of a surgical implant, which method comprises mounting a puncture wall on a hollow body, in a manner such that the hollow body and the puncture wall contribute to defining a chamber designed to be put into communication with the organ, vessel or compartment, the puncture wall is designed to be transpierced by a hollow needle to inject fluid into and/or to tap fluid from the chamber, the method further comprising the following steps:
a) fastening the puncture wall to the hollow body by means of fastener means, in a manner such that the puncture wall, the fastener means and the hollow body form a unitary subassembly;
b) fabricating or supplying a casing that is distinct from the subassembly; and
c) covering the unitary subassembly in the casing, by assembling together the unitary subassembly and the casing, in a manner such that the casing substantially overlies the puncture wall.

### BRIEF DESCRIPTION OF THE DRAWINGS

Various aspects of the present disclosure are described hereinbelow with reference to the accompanying figures.

FIG. 1 is a plan view of one exemplary embodiment of an implantable device of the present disclosure, the device being associated with a catheter lock;

FIG. 2 is a section view on A-A of the device shown in FIG. 1;

FIG. 3 is an overall perspective view of the device shown in FIGS. 1 and 2 without the lock;

FIG. 4 is an exploded diagrammatic section view of the make-up of the subassembly formed by the puncture wall, by the hollow body, and by the means for fastening the puncture wall to the hollow body;

FIG. 5 is a plan view of the subassembly shown in FIG. 4 in its operational state;

FIG. 6 is an overall perspective view of the casing of a device of the present disclosure;

FIG. 7 is a perspective view from below of the casing shown in FIG. 6;

FIG. 8 is a section view of the casing shown in FIGS. 6 and 7;

FIG. 9 is an overall perspective view of the lock shown in FIG. 1; and

FIG. 10 is a diagrammatic section view on B-B of the lock shown in FIG. 9.

### DETAILED DESCRIPTION

The present disclosure relates to an implantable device 1 for injecting and/or tapping a fluid. Such a device, which can also be referred to as an “implantable site”, is designed to be implanted, e.g., surgically, into the body of a patient and, in particular, under the skin of the patient, with a view to constituting an access port for inserting or for extracting fluid substances into or from the body of the patient, who can be a human or an animal.

The implantable device 1 of the present disclosure can be implemented and adapted for various uses.

Firstly, the implantable device 1 of the present disclosure can be designed for injecting fluid into and/or for tapping fluid from an organ or vessel of the body of a patient and, in particular, the venous and/or arterial system of the patient. In such a use, which is known per se, the device 1 makes it possible, for example, to inject liquid medicinal substances into a vein or artery.

The device 1 of the present disclosure can also be adapted to feed implanted reservoirs, of the insulin pump or analgesic pump types.

The implantable device 1 of the present disclosure can also be adapted to inject fluid into and/or to tap fluid from an inflatable and/or deflatable compartment of a surgical implant and, in particular, of a gastroplasty band designed to treat obesity. Such a gastric band is known per se and is generally formed by a flexible strip designed to be looped back around the stomach and closed substantially in the vicinities of and via its two ends by means of a closure system in order to reduce the diameter of the opening of the stoma. The strip can have an annular compression chamber of adjustable volume connected via a catheter to an implantable device 1, which device makes it possible to adjust the internal volume of the chamber in order to adjust the diametrical expansion thereof. However, the device of the present disclosure can be used to adjust volumes in other surgical implants, such as, for example, artificial sphincters or balloons.

Reference is made below more particularly to a hypodermic device, i.e., a device designed to be positioned just under the skin of the patient. However, the device of the present disclosure can be implanted at other places of the body of the patient and, for example, can be implanted deeper.

In accordance with the present disclosure, the device 1 comprises, in one exemplary embodiment, a hollow body 2 and a puncture wall 3 mounted on the hollow body 2 in a manner such that the hollow body 2 and the puncture wall 3 contribute to defining a chamber 4. The hollow body 2 is advantageously substantially rigid. The hollow body 2 is preferably made of a material that is substantially non-transpierceable by a hollow needle. For example, the hollow body 2 can be based on a material that is substantially rigid and hard, such as a metal material or a plastics material. Preferably, the hollow body 2 is made of titanium. Unlike the hollow body 2, the puncture wall 3 is designed to be transpierceable by a hollow needle with a view to injecting and/or to tapping a fluid (a gas, a liquid or a semi-liquid, depending on the use) into or from the chamber 4. The puncture wall 3 is preferably made of an elastomer material of the silicone type having “healing” properties.

By means of these properties, after the puncture wall 3 has been perforated, the hole corresponding to the perforation closes again automatically so that the leaktightness of the chamber 4 is not degraded. As shown in the figures, the puncture wall 3 is advantageously formed by a thick one-piece membrane based on an elastomer material, e.g., silicone. Implementing such a membrane, commonly referred to as a “septum”, is well known. It is also possible, instead of using a single one-piece block of material, to implement a superposition of layers for forming the puncture wall 3, without going beyond the scope of the present disclosure.

Advantageously, the hollow body 2 has a side wall 2A defining a proximal opening 2B, the puncture wall 3 is fastened to the side wall 2A so as to close off the proximal opening 2B. Preferably, the side wall 2A has a substantially tubular shape, of circular section about an axis X-X′. In other words, as shown in the figures, the side wall 2A can be in the shape of a cylindrical band.

The puncture wall 2 closes the proximal opening 2B in substantially leaktight manner so as to avoid, or at least to limit, any leakage of any fluid present in the chamber 4. For this purpose, it is possible to provide for the puncture wall 3 to be made by inserting a circular block of material of diameter slightly greater than the inside diameter of the side wall 2A by force into the side wall 2A so that the circular block of material exerts an inward compression force on the puncture wall 3, which force contributes to imparting self-healing properties to the puncture wall. In other words, the puncture wall 3 advantageously has the characteristic of being self-healing, i.e., it continues to be leaktight after a hollow needle has transpierced it and has then been withdrawn from it.

Naturally, it is possible, without going beyond the scope of the present disclosure, to impart self-healing properties to the puncture wall 3 by any other means known to the person skilled in the art.

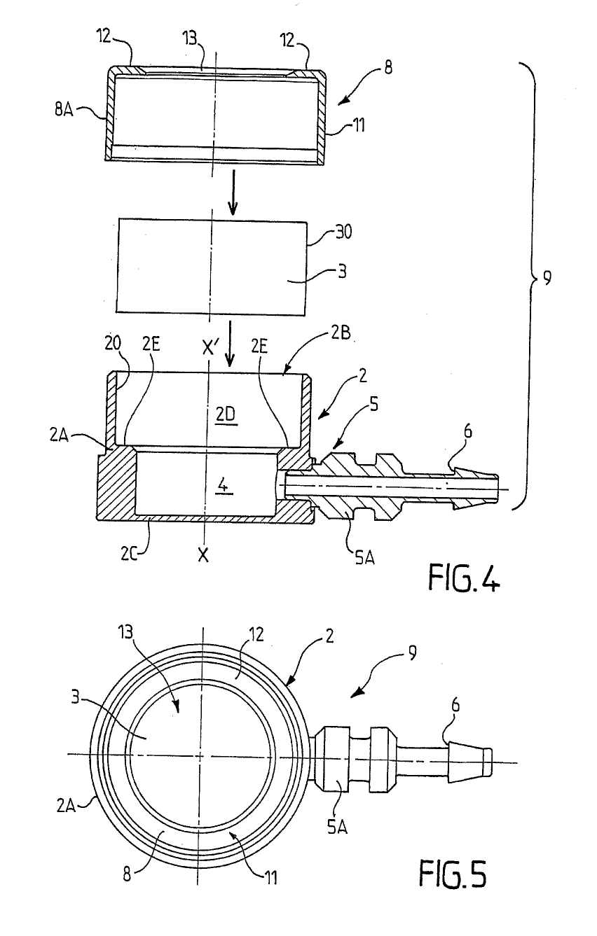

In one exemplary embodiment shown in the figures, and in addition to the side wall 2A, the hollow body 2 further comprises a disk-shaped bottom 2C, from which the side wall 2A extends upwards to the proximal opening 2B. The bottom 2C is then an integral part of the hollow body 2, so that the hollow body 2 is in the general shape of a bowl of substantially U-shaped section (as can be seen in FIG. 4) with its proximal opening 2B being closed off hermetically by the puncture wall 3, in a manner such as to form a substantially leaktight chamber 4. Preferably, as shown, in particular, in FIG. 4, the bottom 2C is formed integrally with the side wall 2A, i.e., the bottom 2C and the side wall 2A are made as one piece, e.g., made from a material that is substantially non-transpierceable, such as titanium. It is naturally possible, without going beyond the scope of the present disclosure, for the bottom 2C, while being an integral part of the hollow body 2, to be mounted on the side wall 2A and fastened thereto by any known means, e.g., by welding. In the variant shown in the figures, the hollow body 2 thus forms a unitary hollow housing that is non-transpierceable and that is provided with a proximal opening 2B closed off in substantially leaktight manner by the puncture wall 3. The chamber 4 is then defined directly by the association of the hollow body 2 and of the puncture wall 3.

In an alternative exemplary embodiment that is not shown but that lies fully within the scope of the present disclosure, the bottom 2C is not part of the hollow body 2 but rather is mounted thereon. For example, the hollow body 2 can be formed solely of the side wall 2A, which defines firstly a proximal opening 2B closed off by the puncture wall 3 and secondly an opposite distal opening. In order to define the chamber 4 completely and in order to close the chamber 4 substantially and hermetically, it is thus necessary, in this exemplary embodiment, to mount a separate bottom on the hollow body 2 so as to close the distal opening and thus to form a chamber 4 that is substantially leaktight. The chamber 4 is then defined by the association of the hollow body 2, the puncture wall 4 and the bottom 2C mounted on the side wall 2A. It can thus be understood that, in this particular exemplary embodiment (not shown), although the hollow body 2 and the puncture wall 3 contribute to defining the chamber 4, they do not define it completely by themselves (unlike in the variant shown), and an additional element (the bottom 2C, in this example) is necessary.

The chamber 4 thus defines an internal volume suitable for receiving a fluid to be injected into and/or to be tapped from the body of the patient. In accordance with the present disclosure, the chamber 4 is designed to be put into communication with the organ, vessel or implant compartment that receives fluid to be injected or that constitutes a source of fluid to be tapped.

To this end, the implantable device 1 advantageously further comprises a duct 5 that puts the chamber 4 into communication with the outside of the device 1.

For example, and as shown in the figures, the duct 5 comprises a pipe 5A of small diameter that is mounted in substantially leaktight manner, e.g., by means of laser welding, into a side orifice provided through the entire thickness of the side wall 2A, at the base thereof, i.e., in the vicinity of the bottom 2C. The pipe 5A thus makes it possible to connect the inside of the housing formed by the hollow body 2 to the puncture wall 4 outside the device 1. Preferably, the pipe 5A is substantially rigid.

The pipe 5A is advantageously made of the same material as the material implemented for forming the hollow body, which material can be titanium, for example. The pipe 5A is preferably designed to be connected to one end of a catheter (not shown), the other end of the catheter is designed to be connected to the organ, vessel or inflatable/deflatable compartment. As is well known per se, the catheter can be inserted by force into the pipe 5A so as to establish the connection between the chamber 4 and the catheter. In order to prevent the catheter and the pipe 5A from disengaging from each other in an untimely manner, the pipe 5A can, in a conventional manner, be provided with anti-disengagement means 6.

For the purpose of further improving the retention of the catheter on the pipe 5A, it is preferable also to implement a lock 7 that is advantageously in the form of a sheath designed to be engaged over the catheter and that compresses the catheter onto the pipe 5A in a manner such that the risk of the catheter escaping from the anti-disengagement means 6 is limited.

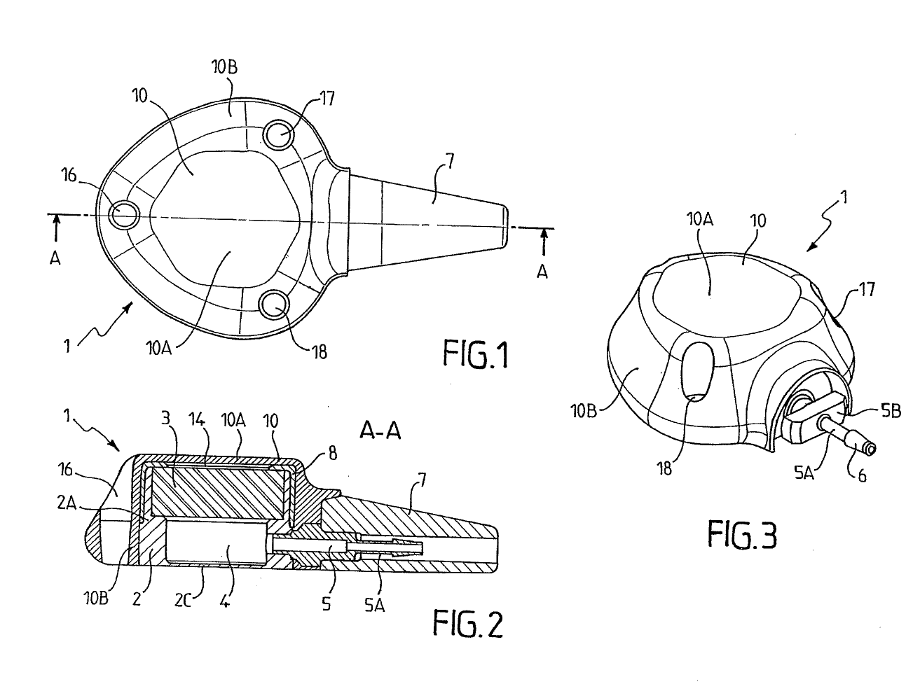

In one exemplary embodiment of the pipe 5A and of the associated lock 7, shown in FIGS. 1-3, 9 and 10 only, the pipe 5A and the lock 7 are provided with complementary blocking means that cooperate to prevent the lock 7 from moving in rotation and in translation relative to the pipe 5A. More particularly, in the example shown in the above-mentioned FIGS., the pipe 5A is provided with a flat 5B, constituted, for example, by a rectangular block shaped projection, the flat being designed to fit into a recess 7A of complementary shape that is provided in the lock 7. The lock 7 is preferably made of a flexible material, such as silicone and is provided with an access opening 7B that gives access to the recess 7A and that is significantly smaller in size than the overall size of the flat 5B, so that the flat must be inserted by force into the lock 7 until it is positioned in the recess 7A.

In accordance with the present disclosure, the implantable device 1 further comprises fastener means 8 for fastening the puncture wall 3 to the hollow body 2 in a manner such that the hollow body 2, the puncture wall 3 and the fastener means 8 together form a unitary subassembly 9. In other words, the fastener means 8, the puncture wall 3 and the hollow body 2 form a one-piece integrated assembly that has its own intrinsic strength. In particular, the function of fastening the puncture wall 3 to the hollow body 2 is performed exclusively by elements internal to the unitary subassembly 9, which elements form the fastener means 8. The fastener means 8 are designed to fasten the puncture wall 3 to the hollow body 2 sufficiently robustly and reliably to withstand the mechanical stresses generated by use of the device 1.

In an exemplary embodiment that corresponds to the exemplary embodiment shown in the figures, the fastener means 8, the puncture wall 3 and the hollow body 2 form an independent unitary subassembly that presents the characteristic of being operational, i.e., the subassembly is directly usable for performing the fluid injection and/or fluid tapping function assigned to the implantable device 1.

Advantageously, and as shown in the figures, the duct 5 is also part of the subassembly 9. It is, however, possible for the duct 5 not to be part of the subassembly 9.

In accordance with the present disclosure, the device 1 further comprises a casing 10 that is distinct from the unitary subassembly 9 and from the fastener means 8.

In other words, the hollow body 2, the puncture wall 3, and the fastener means 8 form a subassembly 9 that is totally independent from the casing 10.

In particular, the cohesion of the hollow body 2, of the puncture wall 3, and of the fastener means 8 is not imparted by the casing 10, but rather solely by means internal to the unitary subassembly 9, namely the fastener means 8, of which subassembly the casing 10 is not part.

Advantageously, the fastener means 8 comprises a blocking element 8A crimped onto the hollow body 2 in a manner such as to block the puncture wall 3 in position. In the exemplary embodiment shown in the figures, and in particular in FIG. 4, the side wall 2A has, as mentioned above, an annular general shape that defines an internal volume 2D of which the chamber 4 is part. The side wall 2A is advantageously provided with an internal shoulder 2E forming a support for the puncture wall 3, which puncture wall is inserted into the internal volume 2D and comes into abutment against the shoulder 2E. In other words, the puncture wall 2 is driven into the side wall 2A and rests on and against the section constriction formed by the inwardly extending shoulder 2E. The fastener means 8 advantageously comprise, as a crimping element 8A, a band 11 provided with an inwardly extending lip 12 that comes into abutment against the puncture wall 3, the band 11 being crimped against the side wall 2A. In other words, the puncture wall 3 is interposed between two opposite bearing surfaces, namely a first bearing surface constituted by the shoulder 2E, and a second bearing surface constituted by the inwardly extending lip 12 of the band 11.

The band 11 is advantageously in the shape of a bowl of substantially U-shaped cross-section, as shown in FIG. 4. More particularly, the end wall of the bowl is advantageously substantially disk-shaped and has a circular central orifice 13 occupying the majority of the area of the end wall.

The material around the central orifice 13 forms the inwardly extending lip 12, which lip is annular in shape. From the bowl end wall there extends a side wall whose free end defines an opening of diameter slightly greater than the diameter of the side wall 2A of the hollow body 2, so that the band 11 can be engaged by force over the hollow body 2, thereby holding the puncture wall 2 captive firmly and reliably, while also giving access to the puncture wall via the orifice 13. The band 11 is advantageously made of the same material as the material forming the hollow body 2, which material is advantageously a metal material such as titanium, for example.

It is naturally possible, without going beyond the scope of the present disclosure, to implement fastener means 8 that are different from the means shown in the figures and based essentially on mechanical assembly (crimping, in this example).

For example, and by way of an alternative, it is possible to implement fastener means 8 that involve molding the puncture wall 3 or adhesively bonding the puncture wall 3 to the hollow body 2. For example, it is advantageous, preferably in addition to the above-mentioned mechanical fastening by crimping, to dispose an adhesive layer that is preferably substantially uniform between the inside face 20 of the side wall 2A and the side wall 30 of the puncture wall 3, with a view to securing excellent peripheral holding of the puncture wall 3 in the hollow body 2.

In accordance with the present disclosure, the casing covers the subassembly 9 so as to overlie the puncture wall 3, the subassembly 9 and the casing being assembled together to form the device 1.

In other words, the casing 10 forms a surface covering for all or part of the subassembly 9, the casing 10 overlying the puncture wall 3 substantially entirely, as can be seen, in particular, in FIG. 2.

Preferably, and as shown in the figures, the casing 10 is provided with suture holes 16, 17, 18 extending through the casing 10 and disposed at its periphery, which suture holes are designed to enable the device 1 to be fastened to the subcutaneous tissue by suturing.

As explained above, the casing 10 is assembled to the subassembly 9, i.e., the casing 10 exists prior to the covering operation, unlike in the prior art in which a casing is deposited by overmolding directly over the hollow body and over the puncture wall.

The device 1 thus makes it possible to coat or to encase the subassembly 9 so as to make it atraumatic, while removing the need to implement an overmolding operation, and without this being detrimental to the strength with which the puncture wall 3 is held relative to the hollow body 2.

Advantageously, the casing 10 is itself a one-piece unit, i.e., it is preferably made as a single piece, e.g., by injection-molding an elastomer material such as silicone, for example. The casing 10 is thus also preferably flexible and therefore atraumatic. The present disclosure thus implements, as described above, assembly of two distinct one-piece subassemblies, each of which exists separately prior to being united with the other, namely the casing 10 and the unitary subassembly 9.

Advantageously, and as shown in the figures, the casing 10 comprises a top membrane 10A substantially overlying the puncture wall 3, i.e., substantially fully masking the puncture wall 3. The casing 10 advantageously further comprises a skirt 10B extending downwards from the top membrane 10A and extending against the hollow body 2, for example, as shown in the figures, substantially along the entire side wall 2A of the hollow body 2.

Advantageously, and as shown in the figures, the skirt 10B is formed integrally with the top membrane 10A.

In the example shown in the figures, the casing 10 overlies the subassembly 9 substantially continuously, except for the bottom 2C of the hollow body 2, which is left directly in contact with the outside, as is the pipe 5A (for obvious operational reasons). However, it is possible, without going beyond the scope of the present disclosure for the casing also to overlie the bottom 2C.

Advantageously, and as shown in the FIGS., the casing 10 defines a recess 10C into which the subassembly 9 formed by the hollow body 2, by the puncture wall 3, and by the fastener means 8 is inserted. Advantageously, the recess 10C defined by the casing 10 has a shape complementary to the shape of the subassembly 9, so that the subassembly 9 fits snugly into the recess, the casing 10 hugging the subassembly 9, as shown in FIG. 2. The subassembly 9 is thus engaged in snug-fitting manner into the casing 10. In other words, the casing 10 caps the sub-assembly 9. As can be seen, in particular in FIG. 2, the top membrane 10A extends substantially parallel to the puncture wall 3, and is superposed thereon. Naturally, the top membrane 10A is designed to be transpierced by the hollow needle in a manner such that the needle can then pass through the puncture wall 3 to reach the chamber 4.

Advantageously, the device 1 is provided with an adhesive interposed between the casing 10 and the puncture wall 3 so as to bond the puncture wall 3 to the casing 10. Preferably, the adhesive is disposed at the interface 14 between the top membrane 10A and the puncture wall 3. The adhesive can, for example, be in the form of a layer that substantially uniformly overlies the puncture wall 3 at the interface 14.

Naturally, the adhesive can alternatively be disposed locally as a spot at the interface 14. It is also possible to leave the interface 14 free of any means for bonding between the top membrane 10A and the puncture wall 3. However, it is preferable for bonding means such as a layer of adhesive to be implemented between the top membrane 10A and the puncture wall 3 because such bonding means make it possible to increase the overall strength and, therefore, the reliability of the device.

In an exemplary embodiment, a layer for absorbing the fluid to be injected and/or tapped is interposed between the puncture wall 3 and the casing 10, in particular, at the interface 14 between the top membrane 10A and the puncture wall 3, in order to limit any leakage of fluid from the chamber 4. It is thus possible to dispose a layer of hydrophilic material at the interface 14 so that, even if the self-healing capacity of the puncture wall 3 is degraded, any liquid escaping from the chamber 4 is absorbed by the absorbent layer before it can escape from the device 1. By way of example, the absorption layer can be made of any one of the following substances and of their derivatives: super-absorbent materials, polyvinyl alcohol (PVA) foam, and hydrophilic gels.

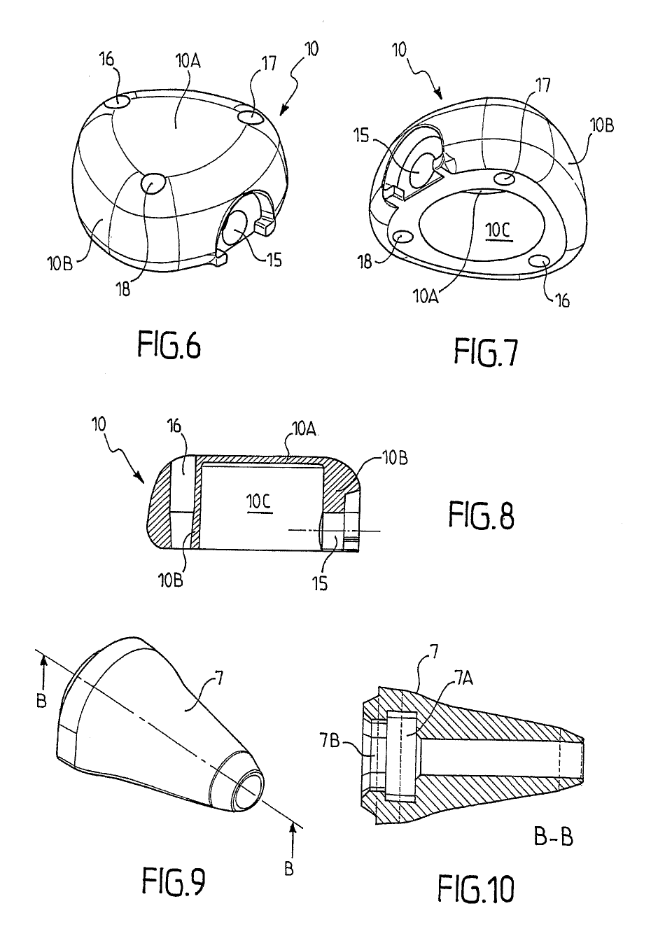

As can be seen in particular in FIGS. 6-8, a side orifice 15 is advantageously provided in the skirt 10B of the casing 10 in order to enable the pipe 5A to pass through. The casing 10 and the subassembly 9 can then be assembled together by inserting the pipe 5A slantwise into the side orifice 15 and then by driving the housing formed by the hollow body 2, the puncture wall 3 and the band 11 into the recess 10C by making use of the flexible and elastic characteristics of the casing 10.

The present disclosure also relates to a method of manufacturing an implantable device 1 for injecting fluid into and/or tapping fluid from either an organ or vessel of the body of a human or animal patient, or else an inflatable and/or deflatable compartment of a surgical implant. The method of the present disclosure can, in particular, be implemented for manufacturing a device 1 of the present disclosure, such as the device that is described hereinabove.

The manufacturing method of the present disclosure comprises mounting a puncture wall 3 on a hollow body 2 in a manner such that the hollow body 2 and the puncture wall 3 contribute to defining a chamber 4 designed to be put into communication with the organ, vessel or inflatable/deflatable compartment, the puncture wall 3 is designed to be transpierced by a hollow needle with a view to injecting fluid into and/or to tapping fluid from the chamber 4.

In accordance with the present disclosure, the method comprises a step a) of fastening the puncture wall 3 to the hollow body 2 by means of fastener means 8, in a manner such that the puncture wall 33, the fastener means 8 and the hollow body 2 form a unitary subassembly 9.

Preferably, the step a) includes a sub-step of mechanically assembling the puncture wall 3 to the hollow body 2. In other words, during the step a), the puncture wall 3 is preferably mechanically secured to the hollow body 2 without any “chemical” process implementing adhesive bonding or overmolding being used for achieving this fastening. As described above, this assembly sub-step can be performed by crimping a band 11 onto the side wall 2A of the hollow body 2.

However, it is quite possible to bond the puncture wall 3 adhesively to the hollow body 2, as described above, without going beyond the scope of the present disclosure. In which case, the step a) includes a sub-step a′) of bonding the puncture wall 3 adhesively to the hollow body 2, which sub-step a′) is performed in addition to or instead of the mechanical assembly sub-step.

In accordance with the present disclosure, the method further comprises a step b) of fabricating or of supplying a casing 10 that is distinct from the sub-assembly 9. Preferably, the step b) comprises a single molding operation, enabling the casing 10 to be made as one piece.

In accordance with the present disclosure, the method further comprises a step c) of covering the subassembly 9 with the casing 10, by assembling together the unitary subassembly 9 and the casing 10 in a manner such that the casing 10 substantially overlies the puncture wall 3.

Advantageously, the step c) includes a sub-step of engaging the unitary subassembly 9 into the casing 10. Advantageously, the step c) can also include a sub-step of adhesively bonding the casing 10 to the puncture wall 3. Such engagement and adhesive bonding sub-steps are described, in essence, above.

Advantageously, the steps a) and c) are distinct. In other words, the fastening step a) is not performed by covering the unitary subassembly 9 by means of the casing 10. Each step a) and c) is preferably performed separately, step c) being performed subsequently to step a).

Advantageously, the steps b) and c) are distinct. This means that the casing 10 is not manufactured at the same time as the unitary subassembly 9 is clad, unlike in the prior art in which the casing 10 is overmolded directly over the housing. In the meaning of the present disclosure, steps b) and c) are performed independently, step c) being performed subsequently to step b).

The present disclosure procures an implantable device 1 that presents an excellent atraumatic characteristic because all of its operating components are encapsulated in a covering casing 10 that is preferably made of a flexible material, such as silicone, for example, and that presents a smooth and continuous geometrical shape that is substantially exempt from irregularities that might injure biological tissue.

The device 1 of the present disclosure is also easy and inexpensive to manufacture since the device 1 is based essentially on simple mechanical assembly operations, without any overmolding being necessary. The mechanical assembly operation is made extremely easy by means of the device being subdivided into two unitary one-piece main subassemblies, each of which has its own intrinsic strength.

The invention described in the present disclosure can be used in making and using implantable devices for injecting and/or tapping fluid.

1. An implantable device for injecting fluid into and for tapping fluid from either an organ or vessel of the body of a human or animal patient, or an inflatable or deflatable compartment of a surgical implant, said device comprising:
a) a hollow body;
b) a puncture wall mounted on said hollow body in a manner such that said hollow body and said puncture wall contribute to defining a chamber designed to be put into communication with said organ, vessel, or compartment, said puncture wall being designed to be transpierced by a hollow needle with a view to either injecting fluid into or to tapping fluid from the chamber;
c) fastener means for fastening the puncture wall to the hollow body in a manner such that said hollow body, said puncture wall, and said fastener means form a unitary subassembly; and
d) a casing that is distinct from said subassembly and that covers said subassembly so as substantially to overlie the puncture wall, the subassembly and the casing being assembled together to form the device.

2. The device of claim 1, wherein the casing comprises a one-piece unit.

3. The device of claim 1, wherein the casing comprises a top membrane substantially overlying the puncture wall, and a skirt extending downwards from said top membrane and extending against the hollow body.

4. The device of claim 2, wherein the skirt is formed integrally with the top membrane.

5. The device of claim 1, wherein the casing defines a recess into which the subassembly formed by the hollow body, by the puncture wall, and by the fastener means is inserted.

6. The device of claim 1, having an adhesive interposed between the casing and the puncture wall in order to attach the puncture wall to the casing.

7. The device of claim 1, wherein a fluid absorption layer is interposed between the puncture wall and the casing for the purpose of limiting any leakage of fluid from the chamber.

8. The device of claim 1, wherein the hollow body comprises a side wall defining a proximal opening, the puncture wall is fastened to said side wall so as to close off the proximal opening.

9. The device of claim 8, wherein the hollow body further comprises a bottom from which the side wall extends upwards.

10. The device of claim 8, wherein the side wall is annular in general shape and defines an internal volume, and said side wall is provided with an internal shoulder forming a support for the puncture wall, which puncture wall is inserted into the internal volume and comes into abutment against the shoulder, the fastening means comprising a band provided with an inwardly extending lip that comes into abutment against the puncture wall, said band being crimped onto the side wall.

11. A method for manufacturing an implantable device for injecting fluid into and/or for tapping fluid from either an organ or vessel of the body of a human or animal patient, or an inflatable or deflatable compartment of a surgical implant, the method comprising:
a) mounting a puncture wall on a hollow body in a manner such that said hollow body and said puncture wall contribute to defining a chamber designed to be put into communication with said organ, vessel, or compartment, said puncture wall is designed to be transpierced by a hollow needle with a view to injecting fluid into and/or to tapping fluid from the chamber;
b) fastening the puncture wall to the hollow body by means of fastener means, in a manner such that said puncture wall, said fastener means, and said hollow body form a unitary subassembly;
c) fabricating or supplying a casing that is distinct from said subassembly; and
d) covering said unitary subassembly in said casing, by assembling together said unitary subassembly and said casing, in a manner such that the casing substantially overlies the puncture wall.

12. The method of claim 11, wherein step b) comprises a single molding operation.

13. The method of claim 11, wherein step d) includes a sub-step of engaging said unitary subassembly into the casing.

14. The method of claim 11, wherein step d) includes a sub-step of adhesively bonding the casing to the puncture wall.

15. The method of claim 11, wherein steps a) and d) are distinct from each other.

16. The method of claim 11, wherein steps c) and d) are distinct from each other.

17. The method of claim 11, wherein step b) includes a sub-step of mechanically assembling the puncture wall to the hollow body.
